(19) United States
(12) Patent Application Publication
(10) Pub. No.: US 2019/0348171 A1
(43) Pub. Date: Nov. 14, 2019

(54) ENTERPRISE MOBILE, CLOUD BASED HEALTH CARE RISK ASSESSMENT SYSTEM

(71) Applicants: Ilan Nachmany, Closter, NJ (US); Sumer Mathur, Sterling, VA (US)

(72) Inventors: Ilan Nachmany, Closter, NJ (US); Sumer Mathur, Sterling, VA (US)

(73) Assignee: Healthcare Facility Compliance Corp., Englewood, NJ (US)

(21) Appl. No.: 16/190,908

(22) Filed: Nov. 14, 2018

Related U.S. Application Data
(63) Continuation-in-part of application No. 16/184,891, filed on Nov. 8, 2018.
(60) Provisional application No. 62/583,453, filed on Nov. 8, 2017.

(51) Int. Cl. G16H 40/20; G06Q 10/06

(52) U.S. Cl. CPC G16H 40/20; G06Q 50/26; G06Q 10/0635

(57) ABSTRACT

An invention is afforded for providing ongoing health facility risk assessment. The invention includes subdividing a facility into a plurality of compartments, wherein each compartment defines a portion of the facility. A risk assessment score is assigned and stored in a database to each inspection point. The risk assessment score is a number indicating an importance and potential harm created when the inspection point fails inspection, and wherein each inspection point indicates a requirement of an element of performance (EP). During inspections, a sum of all risk assessment scores associated with failed inspection points within a particular compartment is calculated. When the sum is greater than a predetermined threshold value for the compartment, an Interim Life Safety Measure (ILSM) is issued by selecting a plurality of ILSM actions from a previously stored list and correlating them with the type of deficiencies to generate, document, and validate steps taken as Interim Life Safety Measures.

### CROSS REFERENCE TO RELATED APPLICATIONS

This application is a continuation-in-part of U.S. patent application Ser. No. 16/184,891, filed Nov. 8, 2018, entitled “Enterprise Mobile, Cloud Based Health Care Compliance System,” which claims the benefit of U.S. Provisional Patent Application having Ser. No. 62/583,453, filed on Nov. 8, 2017, and entitled “Enterprise Mobile, Cloud Based Health Care Compliance and Risk Assessment System,” both of which are hereby incorporated by reference in their entirety.

### BACKGROUND OF THE INVENTION

### 1. Field of the Invention

This invention relates generally to health care facility risk assessment, and more specifically, to an enterprise mobile, cloud based health care compliance and risk assessment systems and methods.

### 2. Description of the Related Art

To improve healthcare quality, organizations have been developed to inspect and accredit health care facilities. Examples of such organizations include the Centers for Medicare & Medicaid Services (CMS) or accredited independent, not-for-profit organizations; i.e., The Joint Commission (TJC), Det Norske Veritas (DNV), Center for Improvement in Healthcare Quality (CIHQ) and Healthcare Facilities Accreditation Program (HFAP). For example, the Joint Commission helps to continuously improve health care for the public by evaluating health care organizations, and accredits and certifies more than 21,000 health care organizations and programs in the United States, including hospitals and health care organizations that provide ambulatory and office-based surgery, behavioral health, home health care, laboratory and nursing care center services. This organization dominates the market regulating 15,716 healthcare facilities in the United states, 3959 Hospitals, 848 Nursing care Center 363 Critical Access Hospitals 1941 Ambulatory Care 3017 Behavioral Health 5298 Home Care and 290 Office Based Surgery, a total of 78% of the US healthcare facilities.

Hospitals depend to varying degrees on receipt of Federal funds to meet their increasing budget needs. Along with partial Federal financing of hospitals, specific Federal requirements have been introduced requiring hospitals to demonstrate compliance in order to receive Medicare/Medicaid funding.

To demonstrate compliance, hospitals were initially given two options by Congress: Either submit to a Federal audit, or achieve accreditation from The Joint Commission (originally known as The Joint Commission on Accreditation of Hospitals, and later as The Joint Commission on Accreditation of Healthcare Organizations). Passing an audit by either of these two organizations would give a hospital “Deemed Status” for a 3-year period which would enable them to collect Medicare/Medicaid funds while they hold this status. Not obtaining or loosing this status would prohibit them from collecting this funding while they were not in “Deemed Status”.

Over the years the healthcare environment has gotten far more complex from a regulatory and technology standpoint as well as a medical and business standpoint. As this has occurred, the number of regulations has increased dramatically; the regulations themselves have become far more complex; documentation requirements have greatly increased; yet due to cost constraints, spending on programs and staffing has been continually cut by the healthcare providers.

Currently, hospitals and healthcare facilities lack the ability to conduct risk analysis at the time of the deficiency's discovery, putting pressure on management to conduct risk analysis within a specific mandatory time frame, which may be missed. In addition, incorporating and monitoring multiple Interim Life Safety Measures (ILSM) requirements, which are health and safety measures that were put in place to protect the safety of patients, visitors, and staff who work in hospitals and other healthcare facilities, may not happen on time.

Since construction, inspection and maintenance activities can impact the facility's life safety; regulations were put in place, which dictates specific mandatory actions, based on risk level, and must be done with a specific pre-defined timeframe. Facilities must also develop their own Interim Life Safety Measures (ILSM) policy for the protection, safety and health of patients, in order to alleviate hazards, incorporating measures of their own, together with the regulated mandatory requirements.

When under construction or when a failed inspection accrues, a list of Interim Life Safety Measures needs to be determined. The reason for determining and activating the specific ILSM is to protect the safety of patients, visitors, and those who work in the hospital. They are based on conducting risk analysis, in order to determine which of the ILSM items must be activated and they need to occur within a certain time, from the time of discovery. It is almost impossible for healthcare facility departments to be compliant, at all time, and meet all ILSM compliant requirements.

In view of the forgoing, there is a need for a system that ensures the ILSM requirements for a safe environment for all a hospital's occupants are completed, while ensuring and validating that the required activities are done on time. In addition, the system should ensure a complete documentation audit trail is maintained and all responsible parties are notified, thus providing hospitals with the ability to assure ongoing compliance and reduce cost by replacing their current existing costly manual inspections.

### SUMMARY OF THE INVENTION

Broadly speaking, embodiments of the present invention address these needs by providing systems that ensures Interim Life Safety Measure (ILSM) are completed, while ensuring and validating the required activities are done on time. In one embodiment a method is disclosed for providing ongoing health facility risk assessment. The method includes subdividing a facility into a plurality of compartments, wherein each compartment defines a portion of the facility. A risk assessment score is assigned to each inspection point and stored in a database. The risk assessment score is a number indicating an importance and potential harm created when the inspection point fails inspection. Each inspection point indicates a requirement of an element of performance (EP). During inspections, a sum of all risk assessment scores associated with failed inspection points within a particular compartment is calculated. When the sum is greater than a predetermined threshold value for the compartment, an ILSM is issued that is a health and safety measure to protect individuals at the facility.

In one aspect, the method can include storing in a database a plurality of ILSM actions and a plurality of correlating deficiency items, wherein each ILSM action defines an action related to protecting individuals at the facility, and each correlating deficiency item defines a correlation between a deficiency that may be encountered at the facility and at least one ILSM action. Here, a one or more ILSM actions are selected based on deficiencies related to the failed inspection points and related correlating deficiency items. Then, the ILSM is issued by generating a list of the selected ILSM actions.

In a further aspect, the method can include documenting that a user has completed each issued ILSM for each impacted EP. In addition, the method can include monitoring work orders associated with each ILSM, and generating a notification that an issued ILSM is no longer needed when all work orders associated with the ILSM are closed in the work order system. In some aspects, risk assessment is not performed for a failed inspection point when an assessment score associated with the failed inspection point is zero. Further, risk assessment for a failed inspection point can be delayed for a predetermined amount of time when a “Time to Resolve” option is selected for a failed inspection point. In this case risk assessment for the failed inspection point is performed when the failed inspection point is not resolved within the predefined period of time.

A computer program embodied on a computer readable medium for providing ongoing health facility risk assessment is disclosed in a further embodiment of the present invention. The computer program includes computer instructions that record a plurality of compartments for a facility, wherein each compartment defines a portion of the facility. Computer instructions are also included that store a risk assessment score for each inspection point in a database. As above, the risk assessment score is a number indicating an importance and potential harm created when the inspection point fails inspection, while each inspection point indicates a requirement of an EP. In addition, the computer program includes computer instructions that calculate a sum of all risk assessment scores associated with failed inspection points within a particular compartment, and issue an ILSM from a predefined ILSM database when the sum is greater than a predetermined threshold value for the compartment.

The computer program can include computer instructions that document that a user has completed each issued ILSM for each impacted EP. Similar to above, the computer program can include computer instructions that monitor work orders associated with each ILSM, and generate a notification that an issued ILSM is no longer needed when all work orders associated with the ILSM are closed in the work order system. In one aspect, risk assessment is not performed for a failed inspection point when an assessment score associated with the failed inspection point is zero. Also, computer instructions can be include that delay risk assessment for a failed inspection point for a predetermined amount of time when a “Time to Resolve” option is selected for a failed inspection point, and perform risk assessment for the failed inspection point when the failed inspection point is not resolved within the predefined period of time.

In a further embodiment, a system is disclosed for providing ongoing health facility risk assessment. The system includes a compartment database that stores a plurality of compartments for a facility, wherein each compartment defines a portion of the facility. A risk assessment database is included that stores a risk assessment score for each inspection point, wherein the risk assessment score is a number indicating an importance and potential harm created when the inspection point fails inspection. A risk assessment module is included that calculates a sum of all risk assessment scores associated with failed inspection points within a particular compartment, and an ILSM module is included that issues an ILSM from a predefined ILSM database when the sum is greater than a predetermined threshold value for the compartment. The system can further record an ILSM completed document when a user has completed each issued ILSM for each impacted EP. In one aspect, work orders associated with each ILSM can be monitored and a notification indicating an issued ILSM is no longer needed can be generated when all work orders associated with the ILSM are closed in the work order system. Typically, the system does not perform risk assessment for a failed inspection point when an assessment score associated with the failed inspection point is zero. Further, the system can delay risk assessment for a failed inspection point for a predetermined amount of time when a “Time to Resolve” option is selected for a failed inspection point. Here, risk assessment for the failed inspection point is performed when the failed inspection point is not resolved within the predefined period of time.

In this manner, embodiments of the present invention provide warnings and alarms when deadlines approach, give emergency notification to users when deadlines have not been met, and provide ongoing, comprehensive regulatory compliance reports to assist in the management of these programs. Further, embodiments of the present invention ensure Interim Life Safety Measures are completed, while also ensuring and validating the required activities are done on time. Other aspects and advantages of the invention will become apparent from the following detailed description, taken in conjunction with the accompanying drawings, illustrating by way of example the principles of the invention.

### BRIEF DESCRIPTION OF THE DRAWINGS

The invention, together with further advantages thereof, may best be understood by reference to the following description taken in conjunction with the accompanying drawings in which:

FIG. 1 is a block diagram showing an enterprise mobile, cloud based compliance system to increase hospital facility management compliance and to improve management and maintenance of healthcare facilities, in accordance with an embodiment of the present invention;

FIG. 2 is a flowchart showing an inspection method for compliance and risk assessment, in accordance with an embodiment of the present invention;

FIG. 3 is a block diagram showing elements of a compliance and risk assessment computer program, in accordance with an embodiment of the present invention;

FIG. 4 is a flowchart showing a process for risk assessment in a compliance system, in accordance with an embodiment of the present invention; and

FIG. 5 is a flowchart showing a method for issuing an ILSM, in accordance with an embodiment of the present invention.

### DETAILED DESCRIPTION OF THE PREFERRED EMBODIMENTS

Embodiments of the present invention provide a system that affords hospitals the ability to assure ongoing compliance and reduce cost by migrating from their current existing costly manual inspections to using a central repository and tracking solution that helps improve healthcare physical environment quality of service and provides for successful compliance outcomes. Embodiments of the present invention ensure Interim Life Safety Measures (ILSMs) are completed, while also ensuring and validating the required activities are done on time. Thus, embodiments of the present invention schedule regulatory compliance activities around regulatory mandated timelines. Embodiments also provide warnings and alarms when deadlines approach, give emergency notification to users when deadlines have not been met, and provide ongoing, comprehensive regulatory compliance reports to assist in the management of these programs. As a result, hospitals can begin a survey knowing required compliance activities have been completed on time and documented correctly, thus dramatically reducing the probability of being cited overall.

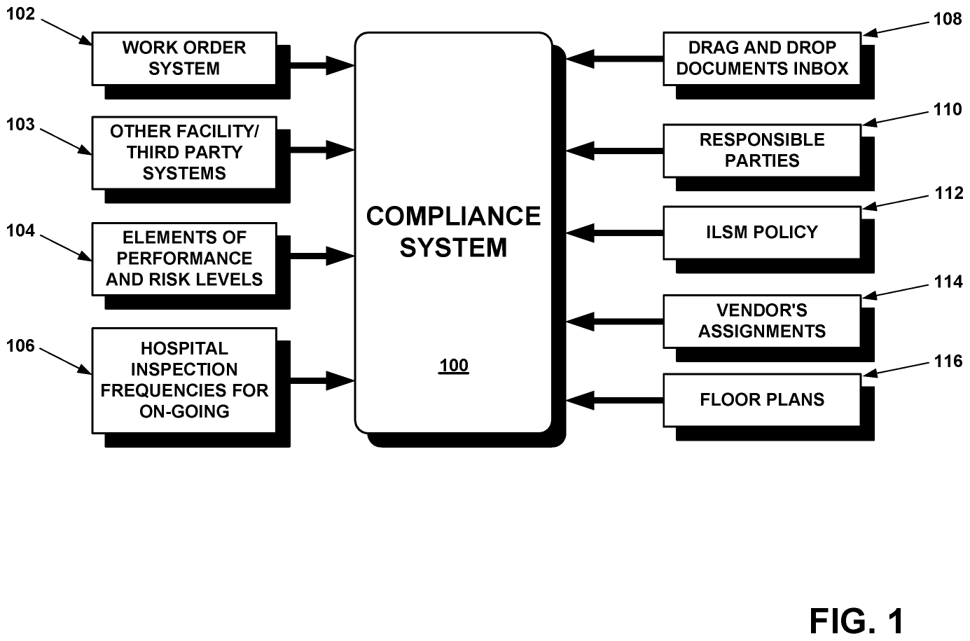

FIG. 1 is a block diagram showing an enterprise mobile, cloud based compliance and risk assessment system 100 to increase hospital facility management compliance and to improve management and maintenance of healthcare facilities, in accordance with an embodiment of the present invention. Embodiments of the present invention integrate with multiple sources of existing data to determine where the facility is in terms of compliance, what needs to be done, and provide the facility a schedule of what needs to be completed. To perform these functions, the compliance system 100 is initially set up to operate in a specific environment as discussed below.

Set up includes integrating the compliance and risk assessment system 100 with a facility's work order system 102, other facility/third party systems 103, providing elements of performance (EP) 104 and associated risk assessment scores based on mandated frequencies and risk levels, providing the hospital's on-going inspection frequencies 106, and setting up an inbox 108 for documents emailed to the compliance system 100. In addition, responsible parties 110 are associated with each EP, an interim life safety measures policy 112 is provided to the compliance system 100, as are vendor assignments 114 and floor plans 116 for the associated facility.

Mandated standards include a plurality of elements of performance (EPs) to indicate what needs to be completed to satisfy the standard. An example EP could be “the fire doors need to be inspected once per year.” Embodiments of the present invention look not only at what frequency an asset should be inspected, but also how the inspection should be done (what should be the inspection points). Each inspection point indicates a requirement of the respective EP. For example, an inspection point can be an asset inspection, a document, or an action to be taken. More specifically, an inspection point is any requirement relating to: assets, machinery, instruments and other facility optional equipment, facility condition inspections, construction status and/or inspections relating to having valid current supporting document, as they relate to environmental care, life safety, and emergency management.

If any of those inspection points is such that risk assessment is required embodiments of the present invention determine whether a life safety measure should be in place. As inspections are done, the associated documentation automatically files in the right place. In addition, if an inspection result requires a work order to be created, the compliance system 100 communicates with the work order system 102 to file a work order into the work order system 102 and then tracks the work order until it is completed. For actions performed by vendors outside the system, the vendors can email their report to the system and the report can be attached to a particular EP.

Embodiments of the present invention can be realized using an enterprise mobility system, wherein users utilize mobile assets, such as tablets and smart phones, to interact with the data and computer programs provided via the embodiments of the present invention. In general, the enterprise mobility nature of the embodiments of the present invention allow for increased productivity and decreased expenses for the facility.

In addition, embodiments of the present invention can be realized via Internet-based computing delivered to the facility's computers and devices through the Internet, often referred to as cloud based computing. This allows embodiments to be accessed and shared as virtual resources in a secure and scalable manner, which further supports enterprise mobility as described above. In some aspects, embodiments of the present invention can be accessed and delivered via the Internet, instead of a local hard drive. In this manner, the related infrastructure of the compliance system 100 can be maintained by a provider, instead of the health facility itself.

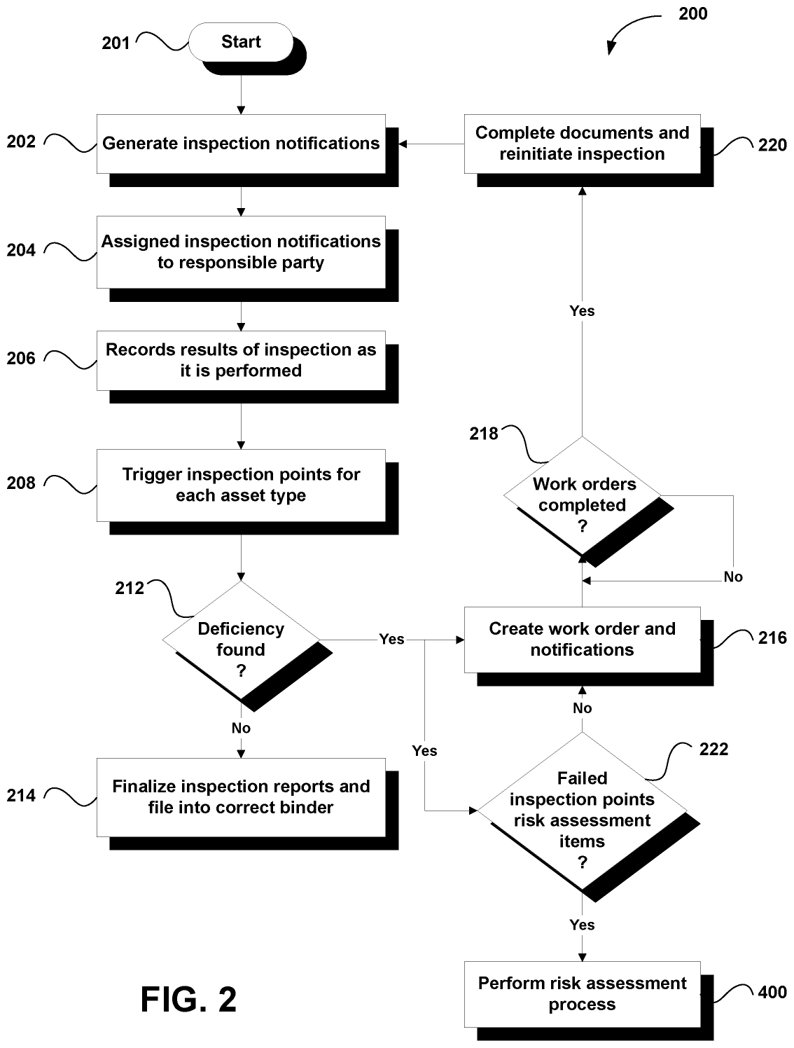

FIG. 2 is a flowchart showing an inspection method 200 for compliance and risk assessment, in accordance with an embodiment of the present invention. In an initial operation 201, initialization and set up operations are performed. Initialization and set up operations can include, for example, integrating the compliance system with a facility's work order system, providing EP and associated risk assessment scores, providing the hospital's on-going inspection frequencies, associating responsible parties with each EP, providing an interim life safety measures policy, determining vendor assignments, and uploading facility floor plans.

In operation 202, inspection notifications are generated and, in operation 204, these notifications are assigned to in-house responsible party or an outside vendor. Based on a predetermined frequency, inspection notifications for EPs are generated by the compliance system. In one embodiment, the compliance system shows the user the standard requirements and the related elements of performance. Beside each EP the user is shown the mandated frequency and the specific person assigned to each EP.

In operation 206, the system records the results of the inspection as it is performed. During the inspection, inspection points are triggered for each asset type, in operation 208. In addition, rounds and drills are recorded with respect to whether they have been performed. Embodiments of the present invention analyze each requirement of the JC to determine if the requirement relates to an asset inspection, a document, or if the requirement relates to a miscellaneous action that must be performed, such as filing rounds that performed every day.

A determination is then made as to whether a deficiency has been found in any inspection point, in operation 212. If no deficiency is found in any inspection point, the method 200 continues to operation 214. Otherwise, the method 200 branches to operation 216 and operation 222.

In operation 214, the inspection reports are finalized and filed into the correct binder. During this operation, the inspection reports are e-signed and the inspection points are reset to the next inspection date. In one embodiment, work from vendors, policies that must be reviewed, etc. are all stored in a repository that is used to show the user whether they have satisfied each requirement. If any requirement is not satisfied, the user can view into the repository and view what is upcoming, what is due, what is past due, where they are now, and how does that affect them in terms of the inspection that will be done by the JC.

In one embodiment, the compliance system has a plurality of binders. When documents are attached to the compliance system, the document is automatically filed into the appropriate binder. Items emailed to the system are sent to the inbox, as indicated in FIG.1. From this inbox, email attachments can be attached to a particular EP. Each EP has its own document requirements indicating what the document needs to include in order to satisfy that EP. These requirements can include such aspects as, for example, a signature and/or a date. When a user attempts to attach a document to an EP, embodiments present a number of questions to the user based on what is required to satisfy the EP's document requirements. In order for the document to be accepted, the user must answer in the affirmative for each question.

If deficiencies are found in any inspection point, the method 200 branches to operation 216 and operation 222. In one embodiment, whenever an inspection point that relates to a risk assessment item fails inspection, the user is given a choice of whether to have “Time to Resolve” or to “Mark as deficiency.” When the user selects “Time to Resolve,” the user is given a predetermined amount of time, for example four hours, to resolve the inspection point deficiency before the inspection point failure result is transferred to operations 216 and 222. If the user resolves the deficiency within the predetermined time period, the inspection point failure is not transferred, and the method moves to operation 214. Otherwise the inspection point failure is transferred to operations 216 and 222. If the user selects “Mark as deficiency,” the inspection point failure is immediately transferred to operations 216 and 222.

In operation 216, a work order and notifications are created. As mentioned previously, embodiments of the present invention are initially integrated with a work order system. In operation 216, the compliance system communicates with the work order system to create one or more work orders to correct the deficiencies found during the inspection. The compliance system then monitors these work orders to determine when they are completed.

In operation 218, a determination is made as to whether the work orders are completed. If the work orders are not completed, the method 200 loops to another operation 218 as it continues to monitor the work orders. If the work orders are completed the method 200 continues to operation 220, where the documents are completed and the inspection is reinitiated.

In addition to performing operation 216, the method 200 branches to operation 222 when deficiencies are found in any inspection point. In operation 222, a decision is made as to whether any of the failed inspection points are risk assessment items. If none of the failed inspection points are risk assessment items, no additional actions are performed and the method continues to generate work orders and monitor them as discussed above with respect to operations 216 and 218. Otherwise, a risk assessment may be performed in operation 400.

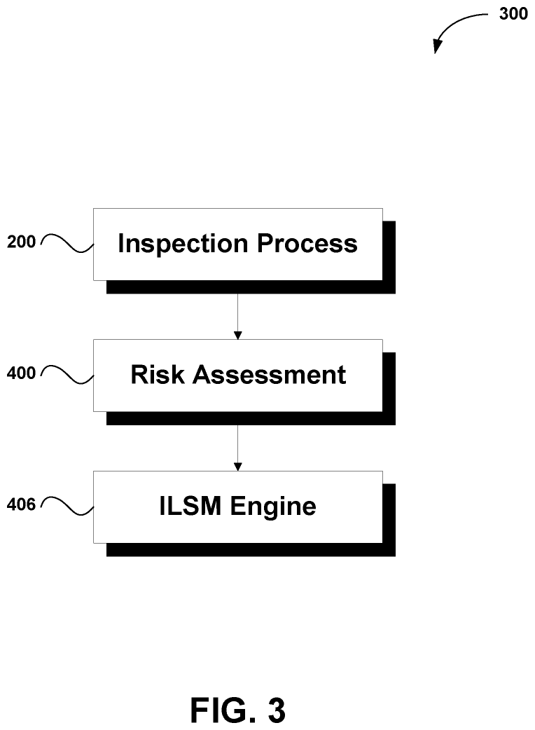

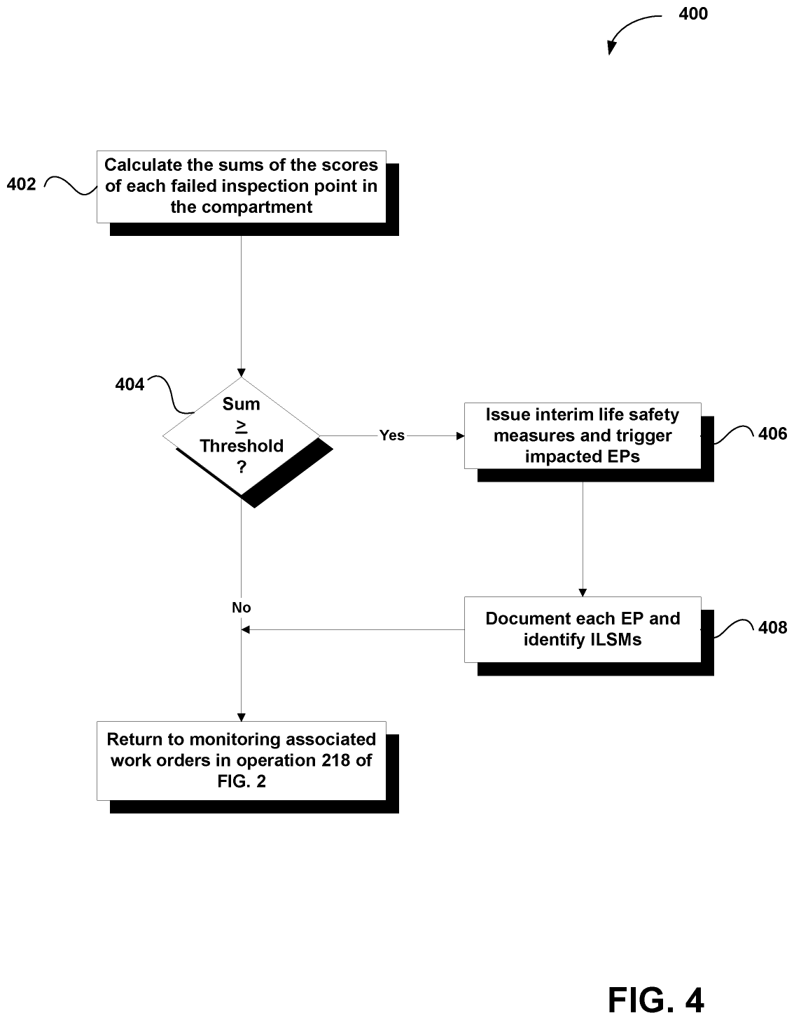

FIG. 3 is a block diagram showing elements of a compliance and risk assessment computer program 300, in accordance with an embodiment of the present invention. The computer program 300 includes computer instructions that perform the inspection method 200 described previously with respect to FIG. 2, computer instructions that perform risk assessment 400 as described with reference to FIG. 4, and an ILSM engine 406 that issues Interim Life Safety Measures (ILSMs).

As noted above, during the inspection method 200, whenever an inspection point that relates to a risk assessment item fails inspection, the user is given a choice of whether to have “Time to Resolve” or to “Mark as deficiency.” When the user selects “Time to Resolve,” the user is given a predetermined amount of time, for example four hours, to resolve the inspection point deficiency before the inspection point failure result is transferred to risk assessment 400. If the user resolves the deficiency within the predetermined time period, the inspection point failure is not transferred to risk assessment 400, otherwise the inspection point failure is transferred to risk assessment 400. If the user selects “Mark as deficiency,” the inspection point failure is immediately transferred to risk assessment process 400. In risk assessment 400, a determination is made as to whether failed points of inspection of the EP data indicate that the inspection point failure should be sent to the ILSM engine 406, where an ILSM is issued if required.

Embodiments of the present invention analyze each specific requirement of the standard ILSM to create the process required within each function. In addition, the facility's own ILSM processes, as determined in their own policy, can be added to the ILSM engine. In addition to creation via assets, ILSMs can also be triggered by events, such as construction. These additional events are determined during set up of the compliance and risk assessment system based on existing standards and the requirements of the particular facility using the invention.

FIG. 4 is a flowchart showing a risk assessment process 400 for a compliance system, in accordance with an embodiment of the present invention. During an inspection, if a deficiency is found in an inspection point that is a risk assessment item, the risk assessment process 400 is performed. In the compliance system, particular inspection points require risk analysis, and thus are given a score based on the importance and the harm created when the inspection point fails inspection.

In operation 402, all deficiencies in a particular compartment are collected and the sums of the scores of each failed inspection point in the compartment is calculated. Each facility is subdivided into a plurality of compartments. These compartments can be of any size, and are usually contained within a single floor of a facility. A compartment can comprise a single room, a portion of a room, a plurality of rooms, or any other combination that facilitates risk assessment for the facility. For example, as mentioned previously, floor plans of the facility are uploaded to the system during initialization. Each floor plan indicates the location of assets on the floor, and shows the location of each compartment that makes up the floor. As assets are inspected, any deficiencies within the compartment are tracked. Deficiencies for items that require risk assessment are collected for each compartment, and the sums of failed risk assessment items scores are calculated for each compartment.

It should be noted that embodiments of the present invention can allow the user to drag and drop assets information into an icon created which can be then dragged and dropped on to the floor plan, select an asset on the floor plan to show all the details for the asset, which comes from the integrated work order system or can be manually added. The user can then inspect the asset, since the compliance system shows all the inspection points that are relevant to that type of asset. For example, a fire door can have certain inspection points and the compliance system shows whether they are in compliance. Some of the inspection points include a score. These inspection points are required to go through a risk assessment in order to determine if interim safety measures are required.

A decision is then made, in operation 404, as to whether the sum of the scores of each failed inspection point in a compartment equals or exceeds a predetermined threshold for the compartment. If the sum does not equal or exceed the predetermined threshold for the compartment, no additional action is required and the method continues to generate work orders and monitor them as discussed above with respect to operations 216 and 218. However, if the sum equals or exceeds the predetermined threshold for the compartment the method 400 branches to operation 406 where one or more interim life safety measure are issued.

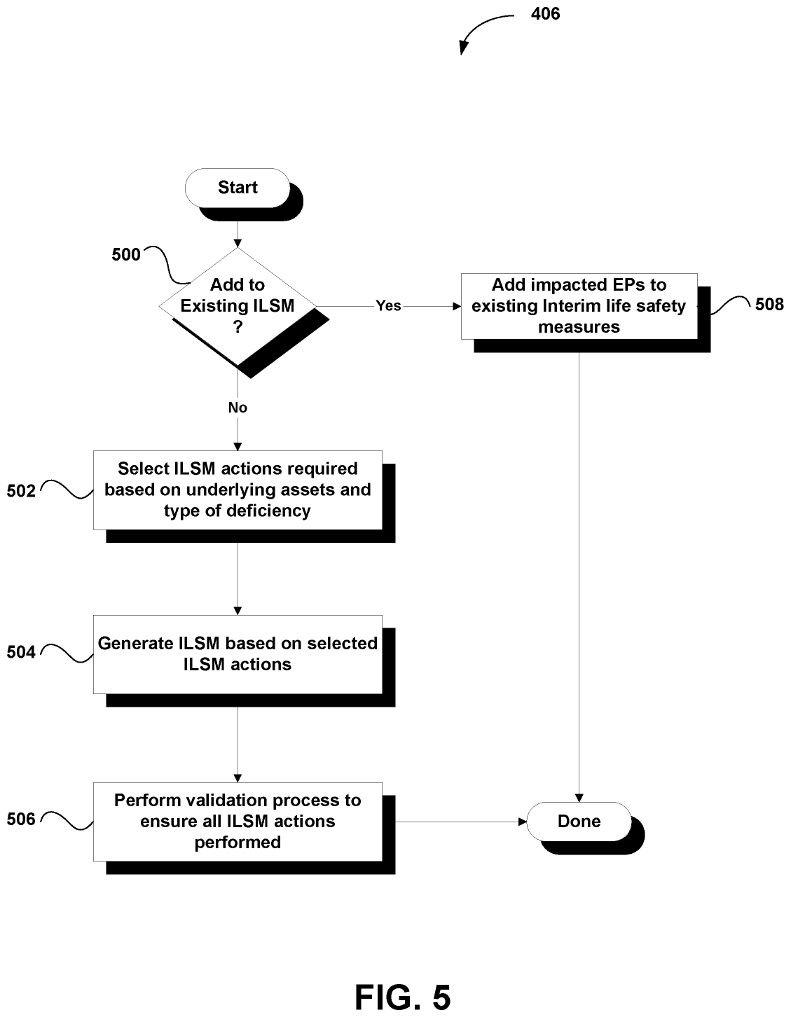

FIG. 5 is a flowchart showing a method 500 for issuing an ILSM, in accordance with an embodiment of the present invention. Because an existing ILSM may already be in place, a determination is made, in operation 500, as to whether to create a new ILSM or add the asset that created the ILSM to an existing ILSM. If a new ILSM is to be generated, the method 500 continues to operation 502, otherwise the method 500 branches to operation 508 where the impacted EPs are added to an existing ILSM.

In operation 502, ILSM actions are selected based on the underlying assets and the type of deficiencies involved. Embodiments of the present invention store in a database a list of ILSM actions defined by accrediting bodies and the particular facility that, when put into action, protect the safety and health of patients by compensating for hazards caused by Life Safety Code deficiencies or construction activity. In addition to the list of ILSM actions, embodiments store in a database correlating relationships between the types of deficiencies that may be encountered and the ILSM actions that address these deficiencies. Embodiments of the present invention then automatically identify and determine, based on the asset deficiencies, ILSM actions to be taken.

Embodiments of the present invention then generate an ILSM based on the selected ILSM actions, in operation 504. The ILSM is a list of selected ILSM actions that are to performed. As noted above, ILSMs are health and safety measures put in place to protect the safety of patients, visitors, and staff who work in the hospital. ILSMs can, for example, take the form of exit signs and pathways to an egress point, fire protection systems including smoke detectors, fire suppression, fire extinguishers and fire alarm systems, smoke barriers, emergency evacuation plans, in addition to many other items that contribute to the well-being and safety of occupants in the hospital or healthcare facility. Construction or maintenance activities can have an impact on the life safety systems in the hospital, thus requiring an Interim plan to address the deficiencies created by the work activity.

When an ILSM is issued, ILSM actions, which are required steps, are selected from a pre-defined list and documented. For example, ILSM actions that may be suggested by ILSM engine might include for example:
(a) Provides temporary but equivalent fire alarm and detection systems for use when a fire system is impaired.
(b) Post signage identifying the location of alternative exits to everyone affected a picture of the posted signage can be taken and each of the item provide for the ability to take pictures and enter notes.
(c) Enforces storage, housekeeping, and debris-removal practices that reduce the building's flammable load
(d) Enforces storage, housekeeping, and debris-removal practices that reduce the building's flammable load
(e) debris removal
(f) Uses temporary construction partitions that are smoke-tight, or made of noncombustible or limited-combustible material that will not contribute to the development or spread of fire.
(g) when the hospital identifies Life Safety Code deficiencies that cannot be immediately the hospital either evacuates the building or
(h) Initiates a fire watch and
(i) Other pre-defined items

In operation 506, a validation process is performed to ensure all ILSM actions are performed. The ILSM engine of the embodiments of the present invention provides the user with the ability to document each ILSM action taken and indicate as performed. Once an ILSM is created, the validation process is activated in order to confirm that all documents and all required action were performed. Alerts are issued if ILSM actions are not performed within the required timeframe. Alerts also are issued at each step of the process, upon deficiency discovery, upon generation of ILSM, and upon actions taken based on ILSM requirements, which provides full documentations of action item and steps taken and are reaffirmed, based on current ILSM incident duration. The ILSM engine also monitors the work order system for completion of all work orders underlying as ILSM incident. Once the work orders are completed, it allows for closing of that ILSM incident, as discussed subsequently.

Referring back to FIG. 4, during an inspection, if an item requires risk assessment the item will have a score. Embodiments of the present invention examine all the items which are risk assessment items, put them in a holding area, and then determine whether all those combined within a particular compartment reach a threshold.

For example, on an exemplary floor within a hospital, there exist a plurality of small compartments. Within one small compartment there may be, for example, one item that has a score of 3 and another item that has a score of 4. In this example, the compartment has a threshold of 6. In this example, since the sum of the failed risk assessment items is 7, which exceeds the compartment threshold of 6, an ILSM is issued. If only one of these are present, then an ILSM is not issued because the sum did not exceed the compartment threshold of 6.

Embodiments of the present invention utilized the fourteen elements of performance mandated by the JC that are required to be addressed. The compliance system of the embodiments of the present invention issues these items, thus requiring the user to address the items. In operation 308, each of these items is required to be documented that “yes” the user has taken an action on the item. Thereafter, the process 400 continues to monitor the associated work orders in operation 218 of FIG. 2. Once those work orders are closed, the deficiencies are closed, and the ILSM is no longer required. Thereafter, the assets are posted again for re-inspection in operation 220 of FIG. 2.

Although the foregoing invention has been described in some detail for purposes of clarity of understanding, it will be apparent that certain changes and modifications may be practiced within the scope of the appended claims. Accordingly, the present embodiments are to be considered as illustrative and not restrictive, and the invention is not to be limited to the details given herein, but may be modified within the scope and equivalents of the appended claims.

What is claimed is:

1. A method for providing ongoing health facility risk assessment, comprising:
subdividing a facility into a plurality of compartments, wherein each compartment defines a portion of the facility;
assigning and storing in a database a risk assessment score to each inspection point, wherein the risk assessment score is a number indicating an importance and potential harm created when the inspection point fails inspection, and wherein each inspection point indicates a requirement of an element of performance (EP);
calculating a sum of all risk assessment scores associated with failed inspection points within a particular compartment; and
issuing an Interim Life Safety Measure (ILSM) when the sum is greater than a predetermined threshold value for the compartment, wherein the ILSM is a health and safety measure to protect individuals at the facility.

2. A method as recited in claim 1, further comprising the operation of storing in a database a plurality of ILSM actions and a plurality of correlating deficiency items, wherein each ILSM action defines an action related to protecting individuals at the facility, and wherein each correlating deficiency item defines a correlation between a deficiency that may be encountered at the facility and at least one ILSM action.

3. A method as recited in claim 2, further comprising the operation of selecting a plurality of the plurality of ILSM actions based on deficiencies related to the failed inspection points and related correlating deficiency items.

4. A method as recited in claim 3, wherein the ILSM is issued by generating a list of the selected ILSM actions.

5. A method as recited in claim 1, further comprising the operation of monitoring work orders associated with each ILSM.

6. A method as recited in claim 5, further comprising the operation of generating a notification that an issued ILSM is no longer needed when all work orders associated with the ILSM are closed in the work order system.

7. A method as recited in claim 1, further comprising the operation documenting a user has completed each issued ILSM for each impacted EP.

8. A computer program embodied on a computer readable medium for providing ongoing health facility risk assessment, comprising:
computer instructions that record a plurality of compartments for a facility, wherein each compartment defines a portion of the facility;
computer instructions that store in a database a risk assessment score for each inspection point, wherein the risk assessment score is a number indicating an importance and potential harm created when the inspection point fails inspection, and wherein each inspection point indicates a requirement of an element of performance (EP);
computer instructions that calculate a sum of all risk assessment scores associated with failed inspection points within a particular compartment; and
computer instructions that issue an Interim Life Safety Measure (ILSM) from a predefined ILSM database when the sum is greater than a predetermined threshold value for the compartment, wherein the ILSM is a health and safety measure to protect individuals at the facility.

9. A computer program as recited in claim 8, further computer instructions that store in a database a plurality of ILSM actions and a plurality of correlating deficiency items, wherein each ILSM action defines an action related to protecting individuals at the facility, and wherein each correlating deficiency item defines a correlation between a deficiency that may be encountered at the facility and at least one ILSM action.

10. A computer program as recited in claim 9, further comprising computer instructions that select a plurality of the plurality of ILSM actions based on deficiencies related to the failed inspection points and related correlating deficiency items.

11. A computer program as recited in claim 8, further comprising computer instructions that generate a notification that an issued ILSM is no longer needed when all work orders associated with the ILSM are closed in the work order system.

12. A computer program as recited in claim 8, wherein risk assessment is not performed for a failed inspection point when an assessment score associated with the failed inspection point is zero.

13. A computer program as recited in claim 8, further comprising computer instructions that delay risk assessment for a failed inspection point for a predetermined amount of time when a “Time to Resolve” option is selected for a failed inspection point.

14. A computer program as recited in claim 13, wherein risk assessment for the failed inspection point is performed when the failed inspection point is not resolved within the predefined period of time.

15. A system for providing ongoing health facility risk assessment, comprising:
a compartment database that stores a plurality of compartments for a facility, wherein each compartment defines a portion of the facility;
a risk assessment database that stores a risk assessment score for each inspection point, wherein the risk assessment score is a number indicating an importance and potential harm created when the inspection point fails inspection, and wherein each inspection point indicates a requirement of an element of performance (EP);
a risk assessment module that calculates a sum of all risk assessment scores associated with failed inspection points within a particular compartment; and
an ILSM module that issues an Interim Life Safety Measure (ILSM) from a predefined ILSM database when the sum is greater than a predetermined threshold value for the compartment, wherein the ILSM is a health and safety measure to protect individuals at the facility.

16. A system as recited in claim 15, wherein an ILSM completed document is recorded when a user has completed each issued ILSM for each impacted EP.

17. A system as recited in claim 15, wherein work orders associated with each ILSM are monitored.

18. A system as recited in claim 17, wherein a notification that an issued ILSM is no longer needed is generated when all work orders associated with the ILSM are closed in the work order system.

19. A system as recited in claim 15, wherein risk assessment is not performed for a failed inspection point when an assessment score associated with the failed inspection point is zero.

20. A system as recited in claim 8, wherein risk assessment for a failed inspection point is delayed for a predetermined amount of time when a “Time to Resolve” option is selected for a failed inspection point, wherein risk assessment for the failed inspection point is performed when the failed inspection point is not resolved within the predefined period of time.
